(19) United States
(12) Patent Application Publication
(10) Pub. No.: US 2019/0078155 A1
(43) Pub. Date: Mar. 14, 2019

(54) METHOD FOR DETERMINING NUCLEOTIDE SEQUENCE

(71) Applicant: UTI Limited Partnership, Calgary (CA)

(72) Inventor: Paul Gordon, Calgary (CA)

(73) Assignee: UTI Limited Partnership, Calgary, AB (CA)

(21) Appl. No.: 15/573,797

(22) PCT Filed: May 14, 2016
(86) PCT No.: PCT/IB2016/052807

§ 371 Date: Nov. 13, 2017

Related U.S. Application Data
(60) Provisional application No. 62/237,437, filed on Oct. 5, 2015.
(60) Provisional application No. 62/161,455, filed on May 14, 2015.

(51) Int. Cl. C12Q 1/6869; G06F 19/10

(52) U.S. Cl. CPC C12Q 1/6869; G06F 19/10

(57) ABSTRACT

The present invention provides a method for determining a nucleotide sequence of at least a portion of an oligonucleotide. In one particular embodiment, the method of the invention uses Dynamic Time Warping and/or additional algorithm to improve both the sensitivity and the speed of nucleotide sequencing.

### CROSS-REFERENCE TO RELATED APPLICATIONS

This application claims the priority benefit of U.S. Provisional Application Nos. 62/161,455, filed May 14, 2015, and 62/237,437, filed Oct. 5, 2015, both of which are incorporated herein by reference in their entirety.

### FIELD OF THE INVENTION

The present invention relates to a method for determining a nucleotide sequence of at least a portion of an oligonucleotide. In particular, the method of the invention includes a signal correction by comparing the obtained signal to a reference signal to more accurately determine the nucleotide sequence.

### BACKGROUND OF THE INVENTION

Single molecule sequencing holds the promise of elucidating genetic data at a speed, price, and physical simplicity unrivalled by today's “next generation DNA sequencing” (NGS). The current crop of NGS machines cost between $10,000's and $1,000,000's, and range in size from a large bread maker to a sedan. In contrast, an early single molecule sequencer called the MinION® (Oxford Nanopore) is the size of a USB key, and is projected to cost around $1,000 when it becomes commercially available. Many kinds of experiments will be enabled or simplified by the exceptionally long reads (e.g., up to 50,000 DNA base) single molecule sequence can produce, if base calling accuracy can be improved.

Therefore, there is a need for a method for significantly improving accuracy of conventional single molecule sequencers.

### SUMMARY OF THE INVENTION

The present invention provides a method for determining single molecule sequences. The method of the present invention is applicable to a wide variety of signal generation, comparison and correction methods. For example, the method of the invention is applicable to the MinION® and any other single molecule sequencing technique due to inherent limitations of accurately observing raw quantitative events at this scale using conventional methods. In particular, some aspects of the invention utilize dynamic time warp (DTW) to correct the generated signal. This corrected signal is then used to compare with reference signal to produce a more accurate sequence reading.

One particular aspect of the invention provides a method for determining a nucleotide sequence (chemically modified or naturally occurring) of at least a portion of an oligonucleotide using one or more of the signal correction methods disclosed herein. Generally, the method includes:
(i) obtaining a signal from a plurality of nucleotides in an oligonucleotide whose nucleotide sequence is to be determined;
(ii) comparing at least a portion of said signal to a corresponding reference signal to determine a signal correction factor; and
(iii) determining a nucleotide sequence of said plurality of nucleotides using said signal correction factor.

The reference signal can be a signal obtained from a synthesized, and typically known or previously determined, nucleotide sequences or it can be a signal obtained from the same species or genes in which the nucleotide sequences are known.

The obtained signal can be separated into a plurality of Quantitative Translocated Events (QTEs) prior to comparison to reference signal. QTEs are then compared to the reference signal to determine the signal correction factor. In some embodiments, the step of comparing the QTEs to the corresponding reference signal includes transforming the obtained signal using Dynamic Time Warping (DTW) to produce a corrected signal, which is then compared to the reference signal. Within these embodiments, in some instances methods of the invention can further include using a streaming variant of DTW to match actual sensor QTEs against expected QTEs for a reference sequence.

In other embodiments, said step of separating said obtained signal comprises separating said obtained signal to an individual nucleotide signal. Still in other embodiments, said corresponding reference signal comprises a signal generated from a single nucleotide or oligonucleotide. Alternatively, said corresponding reference signal comprises a signal generated from a reference oligonucleotide.

Typically, said reference oligonucleotide comprises a predetermined nucleotide sequence having at least 80%, often at least 90%, more often at least 95%, and most often at least 98% of the same nucleotide sequence compared to said oligonucleotide whose nucleotide sequence is to be determined.

Another aspect of the invention provides a method that is schematically illustrated in FIG. 6 below. Briefly, such a method includes matching the obtained signal to reference signals. In such a method, typically the obtained signal is separated or broken into blocks of query signals (or QTEs) to improve the sensitivity of the final match by focusing on collinear subsets of matching query (i.e., reference) signals. Any deviation is than noted as correction factor to allow correction of signal error or signal drift to provide a final nucleotide sequence.

### BRIEF DESCRIPTION OF THE INVENTION

FIG. 1 is an illustrative example of Signal-Sequence Models (SSMs) applicable to a nanopore sequencing device. Multiple quantitative observations of known sequences passing through the device are gathered and used to generate a mean and variance (i.e., reference signal or control signal) for each k-mer (in this case, a 5-mer). In nanopore sequencing, SSMs are used to turn a sensor's quantitative data stream into a predicted DNA sequence.

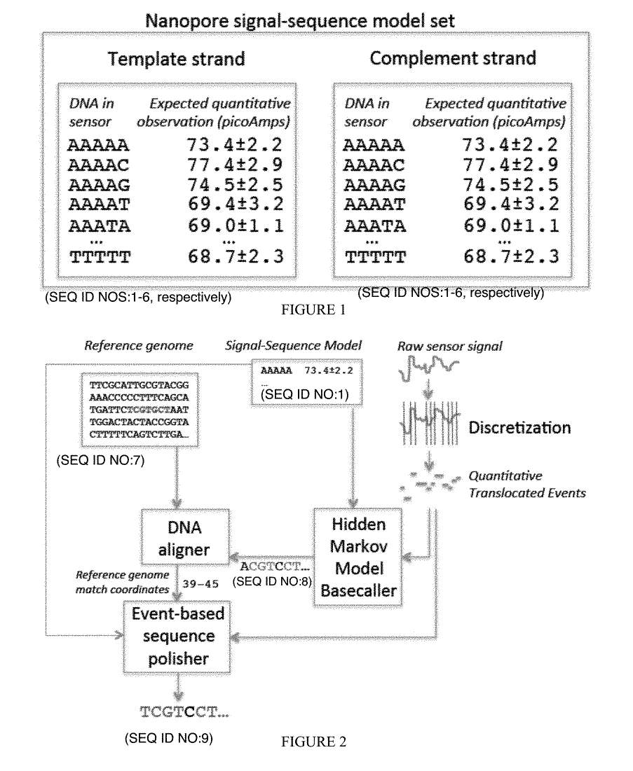

FIG. 2 shows a typical flowchart showing a conventional method for converting a single molecule sensor signal into DNA bases. The raw signal is turned into a set of discrete events representing steady states between DNA translocations in the sensor. These discrete events (QTEs) are used as input to a Hidden Markov Model (HMM), along with existing knowledge of signal expectations (i.e., reference signal) for given DNA k-mers. The HMM produces a DNA sequence that is aligned to a reference genome or a reference oligonucleotide (i.e., reference signal). The alignment & QTEs are used to produce a polished or corrected DNA sequence. In this example, correctly called bases are marked in gray, mistakes are in black. The black A is refined to a T by the polisher or the signal corrector using a correction factor, but the mistaken C remains due to noise in the original signal.

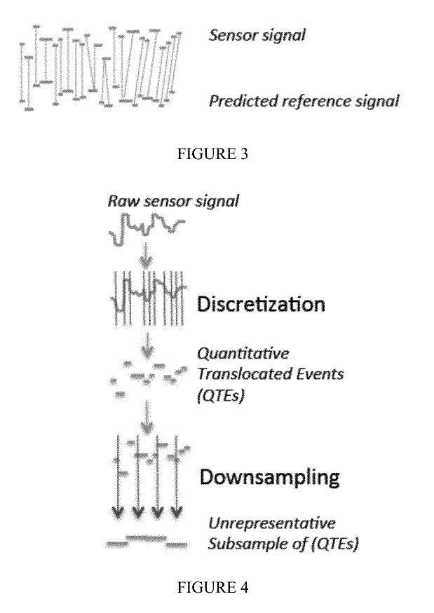

FIG. 3 is an illustrative example of Dynamic Time Warping, where alignment compensates for missing and extra data points by increasing or decreasing the slope of match lines, to minimize overall match distance across the alignment. Limits on and penalization of slope are controlled by a “step policy”.

FIG. 4 shows deficiency in traditional downsampling techniques used to speed up Dynamic Time Warping. In general, traditional downsampling techniques are ineffective for DNA signal due to the high degree of disorder in the discretized event data stream. This is because the evenly spaced subsample is not representative of the whole dataset.

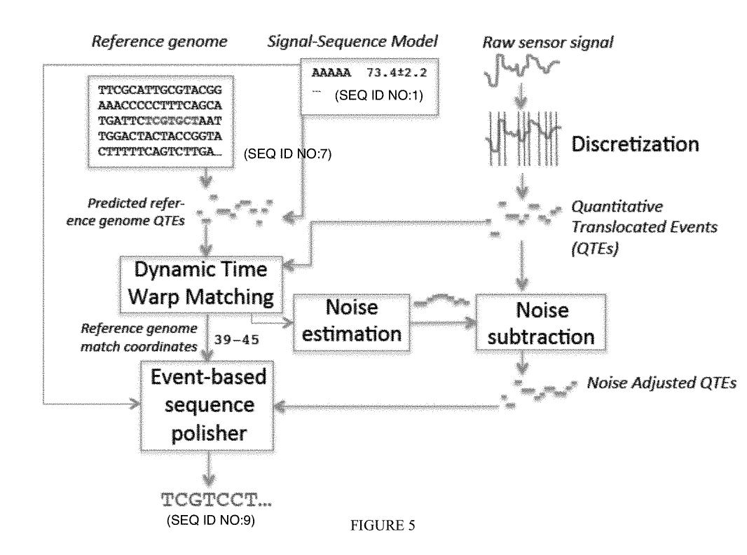

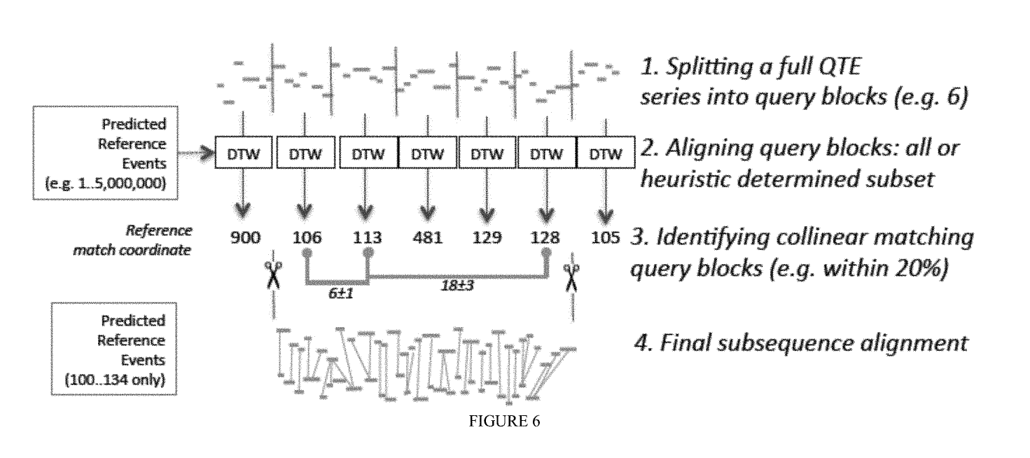

FIG. 5 is a flowchart illustration of one embodiment of the present invention showing conversion of a single molecule sensor signal into DNA bases. A reference genome with which the sample is expected to share significant DNA identity is converted to a set of predicted quantitative events (PQEs) using the Sequence-Signal Model. Raw signal from the sensor is discretized as per usual. The discrete events (QTEs) are aligned to the quantitative reference using Dynamic Time Warping. Noise is estimated from the alignment of quantitative values (picoAmps in the case of nanopores). The QTEs are adjusted for the noise estimate then sent with the alignment to a polisher to produce a final DNA sequence. In this example, the mistaken C from FIG. 2 is corrected through the use of the noise corrected QTEs rather than the original QTEs.

FIG. 6 is an illustrative example showing matching actual observations to reference predictions. Breaking the query into blocks improves the sensitivity of the final match by focusing on collinear subsets of matching query segments.

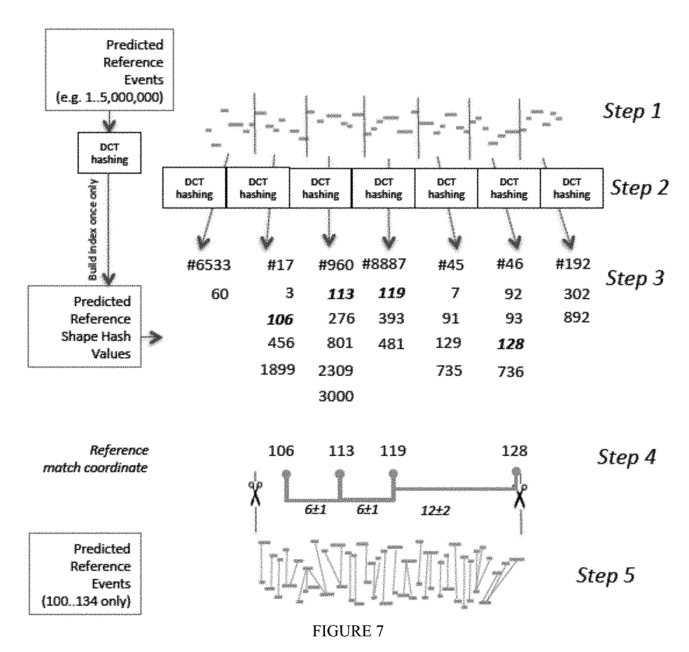

FIG. 7 is illustration of another embodiment of the invention showing collinear matching method using Shape Indices for QTE blocks. This method is based on the Discrete Cosine Transform II (DCT) and finding a collinear path in the set of candidate shape matches for each block. Similar shape indices (hash values) can be made using other discrete transforms such as Fourier and Wavelet. In this figure, step 1 involves splitting a full QTE series into query blocks (e.g. 6); step 2 involves calculating DCT hash value for each query block; step 3 involves retrieving reference locations with the same DCT hash value as the query; step 4 involves identifying collinear matching query blocks, e.g. within 20%; and step 5 involves aligning the signals using DTW for each set of collinear blocks to identify the sequence.

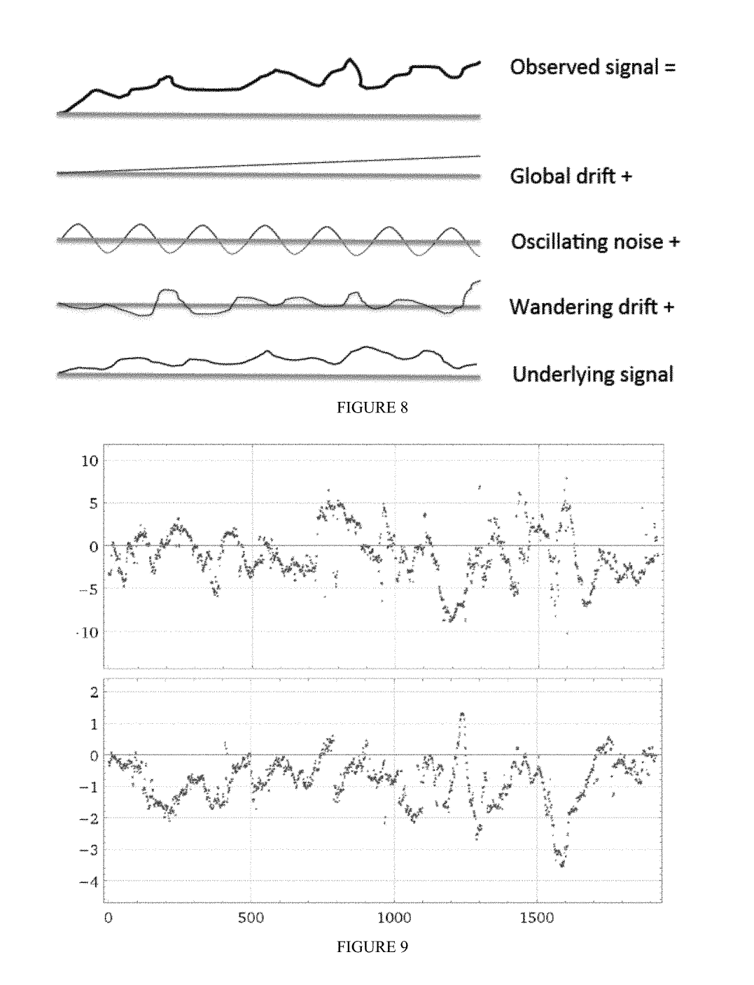

FIG. 8 illustrates that an observed sensor signal may be comprised of underlying signal+noise. Types of signal noise in sensor readings that can be estimated include, but are not limited to, global drift, oscillating noise, and/or wandering drift (given expected observations).

FIG. 9 shows actual wandering drift estimates for a MinION® nanopore sensor using Kernel Density Estimation. Changes in window size and kernel lead to different estimates more or less sensitive to local perturbations. Top: large window and Gaussian kernel. Bottom: small window and Epanechnikov kernel.

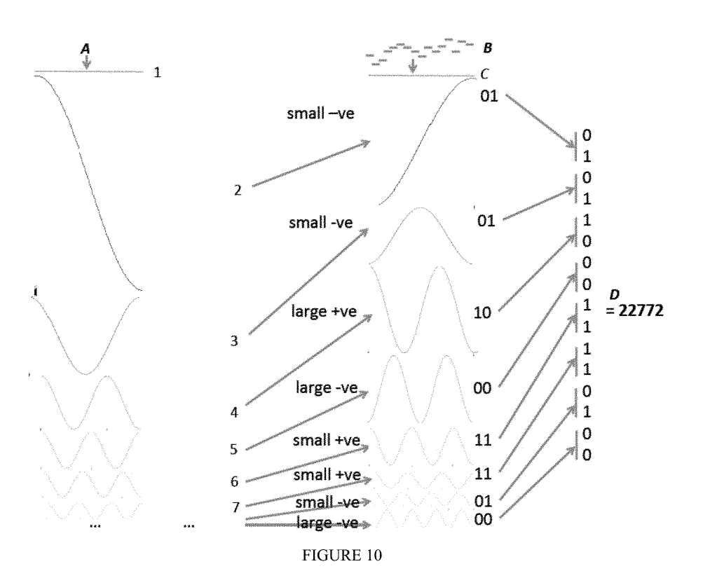

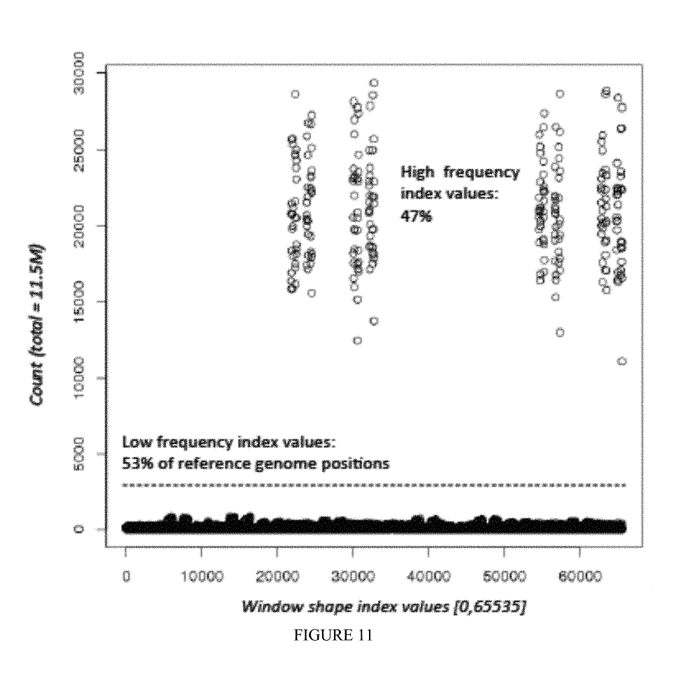

FIG. 10 illustrates one possible shape index calculation, using a 16 event window, and 16 bit value, and the Discrete Cosine Transform II (DCT). Coefficient percentages are actual values for the Influenza A H5N1 genome, using an Oxford Nanopore picoamperage SSM. Legends: A=DCT data for all ref genome 16mers (generates 16 real coefficients); B=DCT on a query 16mer (corrected signal obtained from the sample); 1=1^{st} DCT coefficient (DC constant, e.g., 65 pA, ignored for zero-mean comparisons); 2=2^{nd} DCT coefficient (explains 53% of ref signal on avg); 3=3^{rd} DCT coefficient (19%); 4=4^{th} DCT coefficient (10%); 5=5^{th} DCT coefficient (6%); 6=6^{th} DCT coefficient (4%); and 7=7^{th} DCT coefficient (3%). Small—ve means compared to ref average 1^{st} coefficient, i.e., Interquartile Range Q1-Q2.

FIG. 11 shows actual shape index value frequencies for a Klebsiella pneumoniae genome's reference model picoamperages (11.5M predicted QTEs), using a shape window of 16 picoamperages, and a 16 bit index value (65535 possibilities).

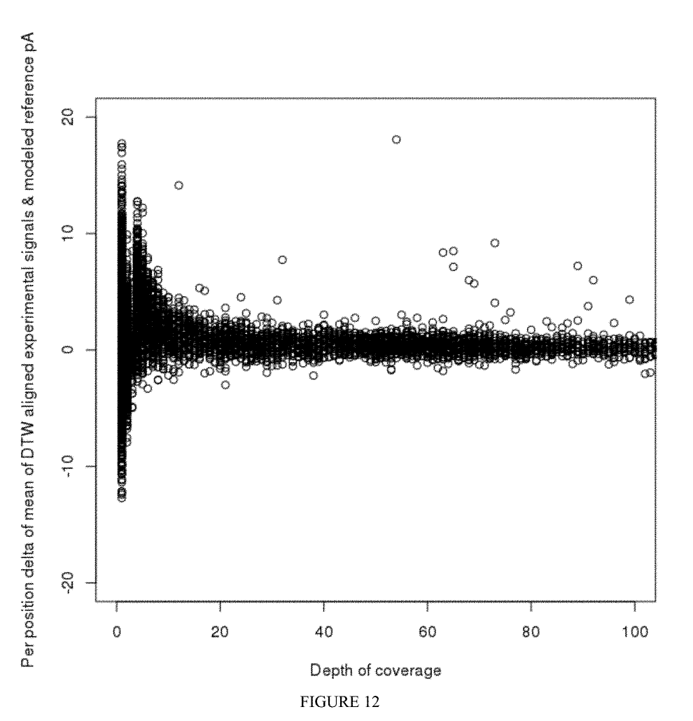

FIG. 12 shows the relationship between the read coverage of a position in part of the lambda phage DNA (MinION® spiked in control DNA) and the difference between the SSM predicted signal and the mean of real signal values aligned to those positions using DTW. Increasing coverage reduces the difference between model and aligned real signal means, therefore creating synthetic signals by averaging real aligned signals leads to improved base calling vs. the individual signals.

### DESCRIPTION OF THE INVENTION

Conventional method, such as Signal-Sequence Model (“SSM”), for determining a single nucleic acid molecule sequencing involves building a model consisting of a mean signal level for each possible distinct nucleic acid input. For example, devices based on conductivity of α-hemolysin nanopores such as Oxford Nanopore's MinION® typically use a pore context of 5 DNA bases such that each of the 1024 permutations of AAAAA, AAAAC, . . . , TTTTT has an expected picoamperage and standard deviation when in the pore. Multiple, distinct SSMs may exist for a device due to changes in context, such as sequencing template or complement nucleic acids, or sequencing chemically modified bases such as methylated DNA. While such a method is useful in sequencing a single nucleic acid molecule, the accuracy of conventional single nucleic acid molecule sequencing is rather poor. The present invention provides a method for significantly increasing the accuracy of SSM method. In some embodiments, the method of the invention provides at least about 10% increase, typically at least about 20% increase and often at least about 30% increase in accuracy rate relative to the conventional SSM method. Alternatively, the method of the invention provides accuracy of at least about 75%, typically at least about 80%, often at least about 85%, and more often at least about 90%. The term “about” when used in conjunction with a numeric value refers to ±20%, typically ±10%, and often ±5% of the numeric value.

It should be appreciated that any current based signal can be used in the method of the invention. An exemplary device that can be used in the method of the invention includes, but are not limited to, Oxford Nanopore's MinION® device. Other currently available and other future devices that are developed can be used with method of the invention. Generally, the invention is directed to a method for improving the accuracy of current SSM method. The present invention will now be described with reference to using Oxford Nanopore's MinION® device and the accompanying drawings. However, it should be appreciated that the scope of the invention is not limited to any particular device.

In some embodiments of the invention, raw nanopore sensor signal, sampled at some given number of Hertz, is divided into a series of discrete events corresponding to a stable, sequence specific picoamperage between each translocation event of nucleic acids in the sensor. A similar approach can be used for any single molecule sensing device. The discrete sequence of quantitative measurement is hereinafter referred to as the Quantitative Translocated Events (QTEs). The QTE is compared to the reference signal (e.g., SSM) to predict the most probable nucleic acid sequence in the sensor. Typically, in the case of the 5 base nanopore SSM, a Hidden Markov Model (“HMM”) is used to resolve 5-mers with very similar picoamperage means. However, in such cases the accuracy for single stranded DNA is typically below 80%. Without being bound by any theory, it is believed that this relatively low accuracy rate is partly due to unmodeled noise in the QTEs.

By constructing an artificial hairpin at one end of the DNA, both strands of the DNA molecule can be observed independently. By building a consensus of both strands' predictions, accuracy can approach up to about 90% as measured by gap-aligning bases to reference sample DNA. Current wet-lab techniques yield between 20-70% hairpinned double strands. Typically, information is lost in almost every step of the state of the art workflow, e.g., by incorrectly parsed QTEs, incorrectly identified hairpins, poor HMM resolution of bases, and/or poor gapped alignment of inaccurate sequence.

Methods of the invention can include bypassing several sources of information loss inherent to the standard QTE/SSM/HMM/DNA/gap-align methodology by instead directly aligning QTEs to reference DNA using Dynamic Time Warping (DTW). DTW computes a match between data points in two time series, and is widely used in fields such as computerized speech recognition to recognize a word in an audio stream despite variable pronunciation length or emphasis (FIG. 3).

The streaming variant of DTW can be used to match actual sensor QTEs against expected QTEs for a reference sequence. Streaming DTW is computationally intensive, and unfortunately, a particular feature of a nucleic acid sensor's signal is that its information content is highly entropic. This entropy means that existing downsampling and data reduction methods for DTW, such as Piecewise Constant Approximation and Wavelets, lose much sensitivity for DNA vs. full signal-query DTW (FIG. 4). The present invention provides a method that allows DTW to work effectively on highly entropic DNA signal data, and models and corrects signal noise.

In one particular aspect of the invention, the method for determining nucleotide sequence comprises: 1) providing or obtaining reference sequence quantitative predictions, 2) matching actual observations to these predictions, and 3) estimating and correcting for observation drift. The corrected observations (i.e., corrected signals) can then be used as more accurate input to existing signal-to-base calling methods. As can be seen, the workflow diagram of this particular embodiment of the invention (FIG. 5) is markedly different from the typical workflow (FIG. 2).

In some embodiments, one or more reference nucleic acid sequences are translated into probable translocation events (PTEs) that would occur if the reference DNA or RNA passed through the sequencing device. This translation of bases to time series signal is accomplished using an existing SSM. See, for example, FIG. 1. If multiple SSMs are applicable to the data, multiple PTE sets are generated as well. FIG. 6. The reference sequences do not need to be exactly the same as the sample sequence.

The method of the invention overcomes the limitations of DTW in matching highly entropic signals such as Quantitative Translocated Events (QTEs) produced by single molecule DNA sequencers in a non-trivial way. An overview or a schematic illustration of one particular method of the invention involving query/reference time series matching process is shown in FIG. 6. A full quantitative observation series from a single molecule sequencer may contain ten of thousands of QTEs. Matching exceptionally long queries is not only very computationally expensive, but fails to find the correct alignment when the QTEs contain significant noise. At least for these reasons, in some embodiments of the invention the query is divided into smaller blocks that can be searched against the reference genome's probable translocation events (PTEs) independently. Different policies can be selected by the user to improve either the sensitivity or speed of the overall process. These include, but are not limited to, the size of the blocks (either uniform, or variable based on an information entropy threshold); overlapping vs. non-overlapping blocks; running all, a random subset, or a geometric pattern of blocks; and using a binary search strategy or other heuristic to prune less informative blocks from needing alignment, or limiting reference search space based on aligned blocks so far.

As an illustrative example, non-overlapping blocks of 64 QTEs works particularly well for MinION data, and a heuristic search provides a major speedup for long, high quality sequences. The heuristic algorithm first aligns distal blocks in the first query half to identify template strand extent. The method then restricts second query half block searches to the same general coordinate region in the reference. In some embodiments, query blocks are aligned to the reference PTEs using a streaming variant of DTW. The search space for optimal alignment can be reduced to speed up to process. Some of the constraints that can be used to increase the rate of query include, but are not limited to, the user selecting between: Sakoe-Chiba band; Ratanamahatana-Keogh band; and Itakura parallelogram. The streaming DTW process returns the location of the best time series match in the reference PTEs, and the normalized distance for the match. In practice, computation acceleration using parallelized hardware such as Graphics Processing Units is useful to cost-effectively process the scale of data produces by single molecule sequencing devices. In one specific embodiment, a Sakoe-Chiba band of 15% is used for MinION® data.

Conventional streaming DTW methods (e.g., UCR Suite, CUDA-DTW) typically rely on quickly calculating successive DTW match lower bound estimates (e.g. LB_{Kim}, LB_{Keough}) across every reference position, followed by full DTW search for the subset of positions meeting the lower bound criteria. This requirement to calculate lower bounds for the entire reference dataset for every query severely restricts the size of genomes that can be searched quickly using DTW. To allow human-sized or larger reference sequences to be searched quickly, some methods of the invention substitute the onerous lower bound calculations in DTW with a form of reference genome indexing. As illustrated in FIG. 7, this indexing (“shape indexing”) is based on the overall shape of expected reference genome picoamperages in a window such as 32 events, or 16 events. Each window of events is transformed using the Discrete Cosine Transform II (DCT), which yields as many coefficients as there are events. These coefficients describe the shape of the event value series at different periodicities. The “energy compaction” effect of the DCT is fundamental to compression schemes such as JPEG encoding for digital images, and is used to assign each reference genome window to a general shape bin with a numeric index (hash) derived from bit encoding the first few DCT coefficients. Typically, the index need only be calculated once, and run against arbitrary many queries. A check for collinearity of query blocks can then be at first restricted to the sites in the genome with the same DCT hash, i.e., same general shape. DCT has the advantage that the first coefficients are resistant to noise in the data, but if collinear match blocks are not found due to high noise, an iterative search can include expanding candidate matches to those with similar hash codes (i.e., the same except for a few low bits which represent small shape contributions).

In other embodiments of the invention, as each query block match is gathered, the cumulative match locations can be scanned for collinearity (i.e., similar order and spacing) with their corresponding query blocks. A user-set limit on the allowed expansion/contraction of the query relative to the reference can be used to control false positives. The minimum and maximum query location within each collinear block set defines the range of each “seed” query-reference subsequence match. For example, a collinearity expansion/contraction limit of 25% for MinION data can be used.

The method of invention can also include re-aligning each seed query-reference subsequence match using global constraints on both the query and reference. In some instances, only a specific subrange of the reference PTEs is aligned. DTW penalization score policies called “step constraints” are applied to control the propensity for insertions and deletions in either the query (QTE) or reference (PTE) sequence to achieve a desired alignment. These step constraint options include, but are not limited to, Symmetric; Asymmetric; and Minimum Variance Matching.

In some embodiments, the user can optionally select to extend the seed alignment. This can be accomplished by prepending the result of an open-beginning DTW alignment, and/or appending the result of an open-end DTW alignment. In either case, the query PTE sequence is comprised of contiguous data points flanking the seed, but not part of another seed. The amount of PTE sequence considered for the alignment extensions can be set by a user policy including, but not limited to, a policy of some percentage deviation from the seed alignment's query-to-reference length ratio.

Where multiple SSMs are applicable to a sequence, the correct SSM (i.e., “reference signal”, and hence sequence context) for a final aligned query segment can be readily determined by the match location in the reference PTEs. In some embodiments, the reference PTEs are a concatenation of the reference genome in each context (each SSM). For example, in the case of MinION® data, one SSM is used to predict template strand DNA bases, and another SSM is used to predict complement strand bases. It follows that query segment matching the first half of the PTEs are template bases, and segments matching the second half of the PTEs are complement bases. This provides a method for identifying hairpin DNA molecules, indicating suitability for template/complement consensus building.

Sensor measurements can be correlated in terms of over/under-estimation relative to the SSM used. This correlation can be split into a time-dependent “global drift”, a predictable oscillating noise, and/or a data neighborhood dependent “wandering drift” effect (FIG. 8). Global drift is well characterized in state of the art signal-to-base callers. Oscillating noise can be estimated using a classic signal processing autoregressive technique such as a Weiner filter. Wandering drift is not well characterized, because it requires expected values from a pre-existing alignment.

For sequencing devices with wandering drift, the final DTW alignment can be used to characterize and determine the magnitude of wandering drift. For example, a difference between each aligned QTE and PTE is calculated, and a standard statistical technique called kernel density estimation (KDE) is applied. In the case of nanopore data, the QTE/PTE difference is picoamperage over/underestimation, which can be represented as ΔpA. KDE is applied across a neighborhood of ΔpAs, with the optimal choice of kernel (Gaussian, Epanechnikov, Uniform, etc.) and neighborhood size (e.g., 8 or 32 data points) depending on the inherent characteristics of wandering drift of the device. For MinION® data, the combination of an Epanichnikov kernel and a neighborhood size of 32 were found to be particularly useful.

The kernel density estimate for each position in the query can be subtracted from the QTE to provide a corrected QTE for downstream base callers. For sequencing devices where wandering drift is correlated across the sensors on the instrument, DTW can be run against the reference sequence of a spiked-in control DNA sample. This allows for drift correction in the absence of a reference genome for the primary sample. As an alternatively to drift correction of individual reads, given the largely random nature of the unmodeled noise component in signals, the uncorrected picoamperage paired to a reference position in each position of the reference genome DTW alignments can be averaged to generate a synthetic composite picoamperage signal. The mean converges on the noise free value of the signal as more reads are mapped to the reference location, and the synthetic signal can be run through the same base caller as original reads were but with a more accurate final base calling due to a less noisy picoamperage dataset. Signal averaging to generate a consensus sequence can also be applied in the absence of a reference signal (i.e., de novo assembly in signal space), using dynamic programming methods to perform multiple signal alignment amongst signal blocks that have been paired using the DTW and/or shape indexing methods outlined herein.

Yet other embodiments of the invention include utilizing standard machine learning techniques such as Expectation Maximization, for example, on a case-by-case basis to determine the optimal settings for each of the user-selected options listed herein, or to splice together different kernel density estimates on a local sequence-region basis.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Experimental Procedure:

Algorithm development and testing was performed on two datasets: E. Coli K12 MG1655 reads (quantitative device results) published by Loman et al. (gigasciencejournal.com/content/3/1/22), and Klebsiella pneumoniae reads done in-house. Both are bacterial genomic samples run for 48 hours using R7.3 chemistry on Oxford Nanopore MinION® devices. A one thousand read sample was randomly chosen from each run, ranging in size from 1000 to 50,000 picoamperage events each.

The reference genomes were converted to predicted quantitative (picoamperage) sensor measurements by a custom Perl programming language script that takes as input 1) a reference DNA sequence, and 2) Oxford Nanopore's 5-mer models, which are included in all OEM results files (i.e., FASTS files). To illustrate, for the K. pneumoniae genome, a 5.2 million base genome was turned into 10.4 million predicted observations for each 5-mer model: 5.2 million forward strand picoamperages, 5.2 million reverse strand picoamperages. Given that two 5-mer models were present in the FASTS file, namely template and complement models, 20.8 million (2×10.4M) events were predicted and saved as a sequence of floating point numbers in a quantitative genome reference file for searching.

Initially, the UCR Suite software (cs.ucr.edu/˜eamonn/UCRsuite.html) was used to perform Dynamic Time Warp matching between complete E. coli reads and the quantitative reference genome. For reads approximately 1000 events in length, the DTW algorithm found accurate matches when the Sakoe-Chiba band parameter was set to 25% or higher.

To improve speed, queries were subdivided into blocks using a custom Perl script and searched against the reference genome in parallel on a 32 multi-core computer. Block sizes of 8, 16, 32, 64, and 128 were explored. Virtually all blocks matched the reference at all sizes, but the vast majority were randomly dispersed in the reference genome, indicating a large number of false positive matches. This was confirmed by DNA sequence level alignment using BWA (bio-bwa.sourceforge.net). A block size of 64 produced the most accurate positives in the E. coli data, with no significant loss of recall down to a Sakoe-Chiba limit of 15%. True positive blocks necessarily have reference genome match locations spaced similarly to their spacing in the query (i.e., collinearity), and these were identified using in the same custom Perl script used to subdivide the query. In some cases, recall of collinearity blocks was lost below 25%, i.e., at a margin 10% higher than the Sakoe-Chiba limit.

Run times for 32-core parallel DTW search of 64-event blocks averaged approximately 15 minutes per read. The search was further accelerated to 10 minutes per read by using a Graphics processing unit (video card) implementation of DTW (github.com/gravitino/cudadtw) and a single CPU. The process was further accelerated to 90 seconds by a heuristic of prioritizing end blocks for first search on the GPUs, and if collinearity was found inner blocks need not be submitted.

Results:

For a sample of 1000 MinION reads from K. pneumoniae, the algorithm identified a genome match for 64% of reads. While the manufacturer's own software (Metrichor) identified 35% of the reads as high quality template+complement (i.e., “2D reads”), the DTW algorithm identified 40% as such. This represents a 5% increase in the number of sequences that can be sent to an extant 2D read polisher for final sequence calling.

With a 1D (template strand only) read polisher, the DTW provides the additional benefit of extremely low false positive rates. In fact, it was found that none of the 293 single strand reads that align to the reference K. pneumonia genome aligned to E. coli K12. In contrast, the best nanopore sequence aligner, called marginAlign, produced E. coli alignments for 25% of these reads.

On real-life genomic data with a window of 16 events, first 8 coefficients of the DCT account for more than 90% of the reference data shape. Preliminary results suggest that for nanopore data a numeric index derived from two-bit encoding the quartile of each coefficient (FIG. 10) is roughly randomly distributed for most hash values (FIG. 11) and corresponds well to DTW lower bounds criteria, making it an effective way to quickly search large reference genomes.

An adaptive indexing scheme was also implemented, wherein the number of bits assigned to a transform coefficient was commensurate with the percentage of energy explained by that coefficient. For example, in MinION® data, the first coefficient explained approximately 53% of the predicted signal in the reference human genome (hg19). In an adaptive 16 bit indexing scheme over a 32 base shape window, 8 bits were therefore assigned to the first coefficient in each (50% of the bits, when rounded down to the nearest bit). The second transform coefficient explained 13%, and was therefore assigned 2 bits. The remaining 6 bits were assigned one each to the third through eighth transform coefficients, all of which contribute less than 6.25% ( 1/16th) of the predicted reference signal energy. For a genome with a different composition, or even a different shape window size in the same genome, the bit assignment might be different. Using the adaptive indexing scheme just described, nearly all high frequency index values disappeared in K. pneumoniae and Homo sapiens indexed genomes except those corresponding to repetitive genome elements.

DTW matches to the spiked in control lambda phage DNA used in the MinION® sequencing kit were examined on a per-reference position basis to determine if deviation from the reference model was mostly position (and hence sequence) specific, or random (noise). FIG. 12 illustrates the relationship between (x) depth of coverage at a position in the lambda reference genome and (y) delta of the SSM model picoamperage and the mean of all signal values aligned by DTW to that position. The trend is to asymptotically approach a delta of zero (mean of real signals=modeled signal from the SSM) with increased coverage, with greatly diminished slope (improvement in accuracy) beyond 20× coverage. In this particular dataset of 238 reads, spanning lambda reference positions 300 to 8300, a “synthetic” read composed of the mean of DTW aligned device signals at each position (no normalization or noise correction applied to the original signal) was run through a recurrent neural network nanopore base caller and achieved 99.775% accuracy for all bases with at least 17× coverage. Remaining sites represent a combination of artifacts due to the chosen DTW step pattern and lambda DNA partially methylated bases (or other chemical modifications). Partial methylation and heterozygosity could be modeled using standard mixture model methods to reduce miscalls in the synthetic read further.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A method for determining a nucleotide sequence of at least a portion of an oligonucleotide comprising:
(i) obtaining a signal from a plurality of nucleotides in an oligonucleotide whose nucleotide sequence is to be determined;
(ii) comparing at least a portion of said signal to a corresponding reference signal using dynamic time warping (DTW) to determine a signal correction factor; and
(iii) determining a nucleotide sequence of said plurality of nucleotides using said signal correction factor.

2. The method according to claim 1, wherein said step (ii) further comprises:
separating said obtained signal into a plurality of Quantitative Translocated Events (QTEs); and
comparing at least a portion of said QTEs to said corresponding reference signal to determine said signal correction factor.

3. The method according to claim 2, wherein said step of comparing said QTEs to said corresponding reference signal comprises comparing said QTEs to said corresponding reference signal using Dynamic Time Warping (DTW).

4. The method according to claim 3 further comprising using a streaming variant of DTW to match actual sensor QTEs against expected QTEs for a reference sequence.

5. The method according to claim 2, wherein said step of separating said obtained signal comprises separating said obtained signal to an individual nucleotide signal.

6. The method according to claim 5, wherein said corresponding reference signal comprises a signal generated from a single nucleotide or oligonucleotide.

7. The method according to claim 5, wherein said corresponding reference signal comprises a signal generated from a reference oligonucleotide.

8. The method according to claim 7, wherein said reference oligonucleotide comprises a predetermined nucleotide sequence having at least 80% of the same nucleotide sequence compared to said oligonucleotide whose nucleotide sequence is to be determined.

9. The method according to claim 8, wherein said reference oligonucleotide comprises a predetermined nucleotide sequence having at least 90% of the same nucleotide sequence compared to said oligonucleotide whose nucleotide sequence is to be determined.

10. The method according to claim 9, wherein said reference oligonucleotide comprises a predetermined nucleotide sequence having at least 95% of the same nucleotide sequence compared to said oligonucleotide whose nucleotide sequence is to be determined.

11. The method of claim 1, wherein said method comprises shape indexing which comprises partitioning expected reference genome picoamperages.

12. The method of claim 11, wherein said shape indexing comprises partitioning expected reference genome picoamperages in a window of at least 8 events.

13. The method of claim 12, wherein said shape indexing comprises an energy compacting transformation of each window of events.

14. The method of claim 13, wherein said energy compacting transformation comprises discrete cosine transformation (“DCT”) II.

15. A method for determining a nucleotide sequence of at least a portion of an oligonucleotide comprising the process outlined in FIG. 6.

16-17. (canceled)

18. A method for determining a sequence of a nucleotide, said method comprising:
(i) obtaining a signal from a single stranded nucleotide;
(ii) comparing at least a portion of said obtained signal to a corresponding reference signal using dynamic time warping (DTW); and
(iii) determining a nucleotide sequence of said single stranded nucleotide using said signal correction factor.

19. The method of claim 18, wherein said single stranded nucleotide is obtained by constructing an artificial hairpin at one end of a double stranded nucleotide to produce said single stranded nucleotide.

20. The method of claim 18, wherein said DTW comprises streaming DTW.

21. The method of claim 18, wherein said obtained signal of said step (i) is segmented into quantitative translocated events (QTEs) prior to said step (ii).

22. The method of claim 18, wherein said obtained signal of said step (i) is discretized prior to said step (ii).

23. (canceled)


SEQUENCE LISTING (9 sequences)

SEQ ID NO: 1 - DNA, 5 bp, Artificial Sequence
  Template 1 DNA Sensor
aaaaa

SEQ ID NO: 2 - DNA, 5 bp, Artificial Sequence
  Template 2 DNA Sensor
aaaac

SEQ ID NO: 3 - DNA, 5 bp, Artificial Sequence
  Template 3 DNA Sensor
aaaag

SEQ ID NO: 4 - DNA, 5 bp, Artificial Sequence
  Template 4 DNA Sensor
aaaat

SEQ ID NO: 5 - DNA, 5 bp, Artificial Sequence
  Template 5 DNA Sensor
aaata

SEQ ID NO: 6 - DNA, 5 bp, Artificial Sequence
  Template 6 DNA Sensor
ttttt

SEQ ID NO: 7 - DNA, 79 bp, Artificial Sequence
  REFERENCE GENOME FOR EXAMPLE
ttcgcattgcgtacggaaaccccctttcagcatgattctcgtgctaattggactactaccggtactttttcagtcttga

SEQ ID NO: 8 - DNA, 7 bp, Artificial Sequence
  MODEL SEQ
acgtcct

SEQ ID NO: 9 - DNA, 7 bp, Artificial Sequence
  EXAMPLE ANALYSIS RESULT
tcgtcct